(19) United States
(12) Patent Application Publication
(10) Pub. No.: US 2022/0192506 A1
(43) Pub. Date: Jun. 23, 2022

(54) SYSTEMS AND METHODS FOR SCREENING INDIVIDUALS FOR AN ELEVATED SKIN TEMPERATURE

(71) Applicant: Kiosk Information Systems, Inc., Louisville, CO (US)

(72) Inventors: Taylor Judson Lightsey, Broomfield, CO (US); Mark William Moyer, Broomfield, CO (US); Joseph John Gronski, Jr., Wheat Ridge, CO (US)

(21) Appl. No.: 17/645,518

(22) Filed: Dec. 22, 2021

Related U.S. Application Data
(60) Provisional application No. 63/128,975, filed on Dec. 22, 2020.

(51) Int. Cl. A61B 5/01; A61B 5/00

(52) U.S. Cl. CPC A61B 5/01; A61B 2562/0257; A61B 5/0077

(57) ABSTRACT

The present disclosure discusses various systems and methods for screening individuals for an elevated skin temperature, including substantially reducing the footprint of conventional thermal imaging system. The footprint of the currently available thermal imaging system is reduced in part because rather than having a blackbody and a person being screened simultaneously located within the thermal camera's field of view, the present disclosure discusses a unique thermal imaging system that provides a reference temperature to the system for subsequent comparison to the thermal camera, using a blackbody, prior to the thermal scanning and imaging the person.

### CROSS-REFERENCE TO RELATED APPLICATIONS

The present application claims priority to and the benefit of U.S. Provisional Application No. 63/128,975, filed on Dec. 22, 2020, the disclosure of which is hereby incorporated by reference in its entirety.

### TECHNICAL FIELD

The present disclosure relates generally to systems and methods for screening individuals for an elevated skin temperature using a thermal imaging device and a blackbody calibration source.

### BACKGROUND

Certain industries, businesses, and government agencies are currently using thermal imaging cameras to screen individuals for elevated skin temperature, which is a potential sign of infection of particular diseases, such as the Coronavirus Disease 2019 (COVID-19). An example of such a thermal imaging system is shown in FIG. 10, which is a reproduction of the thermal imaging room setup depicted on the United States Food & Drug Administration website. As illustrated in FIG. 10, the thermal imaging system is configured to image the head, face, neck or other portion of an individual person ranging from 0.75 to 2.2 meters tall. Specifically, it may be desirable to sense and image the region of the head located medially to the inner canthus of the eye to obtain and meet certain accuracy requirements.

Continuing to refer to FIG. 10, the thermal imaging system may include (i) an infrared thermometer (IRT) disposed a predetermined distance in front of the person, (ii) a low-reflective background to avoid more reflective backgrounds, such as glass, mirrors, metallic surfaces or etc., disposed behind the person and (c) a blackbody disposed at the same predetermined distance that the person is located in front of the IRT. Systems such as this have been manually adjusted or provided minimal or no adjustment, thereby limiting the height range. Alternately, conventional thermal imaging systems have attempted to meet the height requirements by having subjects stand further away, which in turn, potentially increases the field of view of the thermal camera while sacrificing accuracy.

The FDA and International Organization for Standards (ISO) have recommended pairing each IRT or thermal camera with a blackbody simulator as an external temperature reference, which is typically referred to as a blackbody, because incorporating a blackbody into the thermal imaging system may contribute to improving the system's accuracy. A blackbody is an object with a known emissivity of 1, which theoretically means that it absorbs and radiates all thermal energy. In practice, however, a blackbody may only have an emissivity ranging from 0.9 to 0.99, which is sufficient to calibrate a thermal camera. That is, a blackbody may be used as an optical reference source to obtain more accurate thermal measurements. Specifically, the blackbody provides a reference temperature point against which to compare the temperature obtained by IRT to reduce potential drift, variability in the IRT pixel array, or detection errors that may arise during the measurement of a person's skin temperature. An example of a blackbody is the NIGHTINGALE™ Model BTR-03 Black Body Temperature Reference Source.

Referring again to the thermal imaging system shown in FIG. 10, the blackbody is typically disposed in the IRT's field of view along with the person because both the IRT and the person are situated at a predetermined distance in front of the IRT. For example, conventional thermal imaging systems typically locate the blackbody from a few feet to potentially more than ten (10) feet away from the thermal camera or IRT. Including the blackbody the at this range from the IRT and/or at the same distance from the IRT as the person leads to a large footprint for the thermal imaging system. And having a large footprint is undesirable because as, discussed above, accuracy suffers and using a large amount of space encroaches on throughways for people to move through the facility in which the thermal imaging system is located.

Additional shortcomings of the currently available thermal imaging systems include the following: (a) the large footprint causes the blackbody and the subject to be farther away from the thermal camera, and the further away the subject is from the thermal camera, the greater the uncertainty of the accuracy of the reading; (b) unless there is an very or extremely high IRT resolution, it is either unlikely or not possible to meet the FDA target zone of 240 mm height×180 mm width for a subject's facial size, where 1 pixel is equal to or about 1 mm; (c) if the subject is wearing glasses, it is either unlikely or not possible to measure the subject's inner eye canthus, and the temperature measurement will likely need to be taken at a different location and/or with a different system; and (d) if the subject is wearing glasses, the temperature taken of the subject using a the currently available thermal imaging system may produce an inaccurate result.

### SUMMARY

The present disclosure discusses various systems and methods for screening individuals for an elevated skin temperature, and the various systems and methods discussed herein reduce some of the shortcomings associated with conventional systems and methods. For example, the systems and methods disclosed herein substantially reduce the footprint of the thermal imaging system while increasing thermal imaging accuracy. The footprint of the thermal imaging system is reduced in part because the blackbody is omitted from the IRT's field of view of the person, even though the thermal imaging system includes a blackbody. Rather than having the blackbody and the person simultaneously located within the IRT's field, the present disclosure discusses a unique thermal imaging system that provides a reference temperature to the system for subsequent comparison to the IRT. That is, the reference temperature is obtained using a blackbody, but the reference temperature is obtained either prior to or following the IRT scanning and imaging of the person. The thermal imaging system includes a motorized IRT that automatically travels and rotates between a calibration position and an image taking position, whereupon the IRT is directed and focused at the blackbody when the IRT is in the calibration position, and the IRT is directed and focused at the person when the IRT is in the image taking position. For example, depending upon the resolution of the IRT, it may be desirable for the camera to be positioned approximately between 17 and 25 inches (i.e., 17.0, 17.5, 18.0, 18.5, 19.0, 19.5, 20.0, 20.5, 21.0, 21.5, 22.0, 22.5, 23.0, 23.5, 24.5 or 25.0) inches away from the subject's face to meet the FDA target zone requirement of 240 mm height×180 mm width for a subject's facial size, where 1 pixel is equal to or about 1 mm.

Example systems and methods for screening individuals for an elevated skin temperature are as follows.

In an Example 1, a system for scanning an individual's skin temperature, the system comprises: a housing; a blackbody; a camera housing subassembly coupled to the housing, wherein the camera housing subassembly is moveable relative to the blackbody, wherein the camera housing subassembly comprises a proximity sensor and a thermal imaging device; a motorized drive system coupled to the camera housing subassembly, wherein the motorized drive system is configured to (i) move the thermal imaging device toward and away the blackbody along a vertical axis and (ii) rotate the thermal imaging device about a horizontal axis, wherein the horizontal axis is offset substantially 90 degrees from the vertical axis; and non-transitory computer readable medium having a computer program stored thereon for controlling movement of the thermal imaging device and timing at which the thermal imaging device obtains a thermal image of the individual's skin temperature, the computer program comprising instructions for causing one or more processors to: move the thermal imaging device toward the blackbody along the vertical axis and rotate the thermal imaging device about the horizontal axis to a calibrated position; obtain a first thermal image when the thermal imaging device is in the calibrated position and directed toward the blackbody; move the thermal imaging device away from the blackbody along the vertical axis and rotate the thermal imaging device about the horizontal axis to an individual image taking position; obtain a second thermal image when the thermal imaging device is in the individual image taking position and directed toward the individual; and calculating the individual's skin temperature as a function of the second thermal image and the first thermal image.

In an Example 2, the system of Example 1, wherein the computer program further comprises instructions to output the individual's skin temperature.

In an Example 3, the system of Example 1, wherein the motorized drive system is configured to linearly translate the thermal imaging device toward and away the blackbody along the vertical axis.

In an Example 4, the system of Example 3, wherein the computer program further comprises instructions for causing one or more processors to linearly translate the thermal imaging device toward the blackbody along the vertical axis to the calibrated position.

In an Example 5, the system of Example 3, wherein the computer program further comprises instructions for causing one or more processors to linearly translate the thermal imaging device away from the blackbody along the vertical axis to the individual image taking position.

In an Example 6, the system of Example 3, wherein the computer program further comprises instructions to calculate the individual's skin temperature as a function of the second thermal image being calibrated by the first thermal image.

In an Example 7, the system of Example 1, wherein the motorized drive system comprises: a first motor; a threaded rod coupled to the first motor; and a linear slide coupled to the threaded rod and camera housing subassembly, whereupon rotation of the first motor, the threaded rod rotates and the linearly translate the camera housing subassembly toward or away from the blackbody along the vertical axis.

In an Example 8, the system of Example 1, wherein the motorized drive system comprises: a second motor; and a drive train coupled to the second motor and the camera housing subassembly, whereupon rotation of the second motor, the camera housing subassembly rotates about the horizontal axis.

In an Example 9, the system of Example 1, wherein the blackbody is coupled to the housing.

In an Example 10, the system of Example 1, wherein the blackbody is separate from the housing.

In an Example 11, a method for scanning an individual's skin temperature, the method comprising: providing a skin temperature sensing system, the system comprises: a housing; a blackbody coupled to the housing; a camera housing subassembly coupled to the housing, wherein the camera housing subassembly is moveable relative to the blackbody, wherein the camera housing subassembly comprises a proximity sensor and a thermal imaging device; and a motorized drive system coupled to the camera housing subassembly, wherein the motorized drive system is configured to (i) move the thermal imaging device toward and away the blackbody along a vertical axis and (ii) rotate the thermal imaging device about a horizontal axis, wherein the horizontal axis is offset substantially 90 degrees from the vertical axis; and moving the thermal imaging device toward the blackbody along the vertical axis and rotating the thermal imaging device about the horizontal axis to a calibrated position; obtaining a first thermal image when the thermal imaging device is in the calibrated position and directed toward the blackbody; moving the thermal imaging device away from the blackbody along the vertical axis and rotating the thermal imaging device about the horizontal axis to an individual image taking position; obtaining a second thermal image when the thermal imaging device is in the individual image taking position and directed toward the individual; and calculating the individual's skin temperature as a function of the second thermal image and the first thermal image.

In an Example 12, the method of Example 11, further comprising displaying the individual's skin temperature.

In an Example 13, the method of Example 11, wherein the motorized drive system is configured to linearly translate the thermal imaging device toward and away the blackbody along the vertical axis.

In an Example 14, the method of Example 12, wherein moving the thermal imaging device comprises linearly translating the thermal imaging device toward the blackbody along the vertical axis to the calibrated position.

In an Example 15, the method of Example 12, wherein moving the thermal imaging device comprises linearly translating the thermal imaging device toward the blackbody along the vertical axis to the individual image taking position.

In an Example 16, the method of Example 11, further comprising calculating the individual's skin temperature as a function of the second thermal image being calibrated by the first thermal image.

While multiple examples are disclosed, still other embodiments of the present disclosure will become apparent to those skilled in the art from the following detailed description, which shows and describes illustrative examples of the disclosure. Accordingly, the drawings and detailed description are to be regarded as illustrative in nature and not restrictive.

### BRIEF DESCRIPTION OF THE DRAWINGS

FIG. 1 is an illustration of a perspective view of an example a temperature screening workstation, in accordance with an embodiment of the present disclosure.

FIG. 2 is an illustration of alternative perspective view of the example the temperature screening workstation depicted in FIG. 1, in accordance with an embodiment of the present disclosure.

FIG. 3 is an illustration of a thermal scanning device and a calibration body of the example the screening workstation depicted in FIG. 1, wherein the thermal scanning device is oriented in a calibration position, in accordance with an embodiment of the present disclosure.

FIG. 4 is an illustration of a thermal scanning device and a calibration body of the example the screening workstation depicted in FIG. 1, wherein the thermal scanning device is oriented in an image taking position, in accordance with an embodiment of the present disclosure.

FIG. 5 is an illustration of a front view of a thermal scanning device and a calibration body of the example the screening workstation depicted in FIG. 1, wherein the drive system for the thermal scanning device is exposed, in accordance with an embodiment of the present disclosure.

FIG. 6A is an illustration of an alternative front view of a thermal scanning device and a calibration body of the example the screening workstation depicted in FIG. 1, wherein the drive system for the thermal scanning device is exposed, in accordance with an embodiment of the present disclosure.

FIG. 6B is an illustration of an alternative front view of a thermal scanning device of the example the screening workstation depicted in FIG. 1, wherein the drive system for the thermal scanning device is exposed, in accordance with an embodiment of the present disclosure.

FIG. 7 is an illustration of a perspective view of a thermal scanning device of the example the screening workstation depicted in FIG. 1, wherein the drive system for the thermal scanning device is exposed, in accordance with an embodiment of the present disclosure.

FIG. 8 is an illustration of a block diagram of an example workstation having a computer system which may be used to implement all or certain or a combination of the methods illustrated in FIG. 9 and/or implement all or certain or a combination of aspects of the examples discussed herein.

FIG. 9 is an illustration of a flow diagram for a method of screening a plurality of individuals, in accordance with an embodiment of the present disclosure.

FIG. 10 is a depiction of an example of a known prior art thermal imaging system.

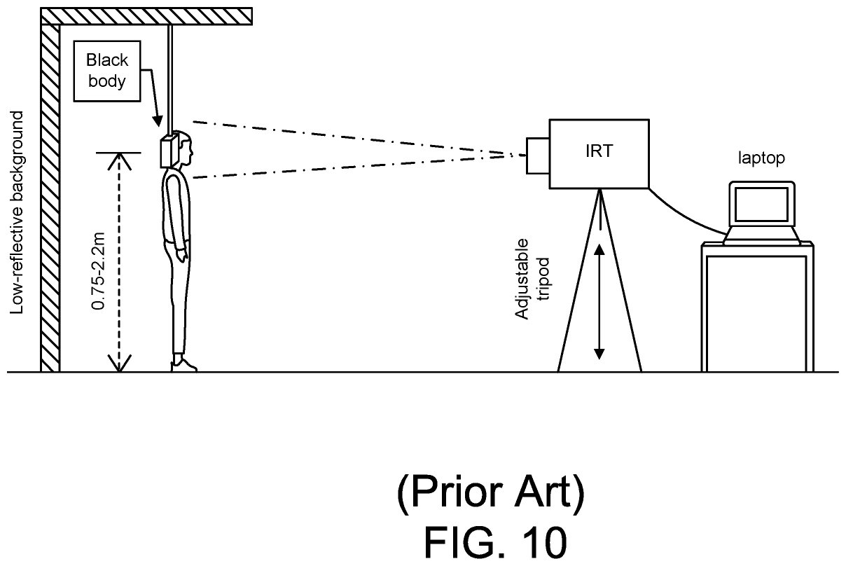

FIG. 11 is an illustration of a perspective view of an example a temperature screening workstation, in accordance with another embodiment of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

As set forth above, examples disclosed herein, may reduce some of the shortcomings associated with conventional thermal imaging systems and methods.

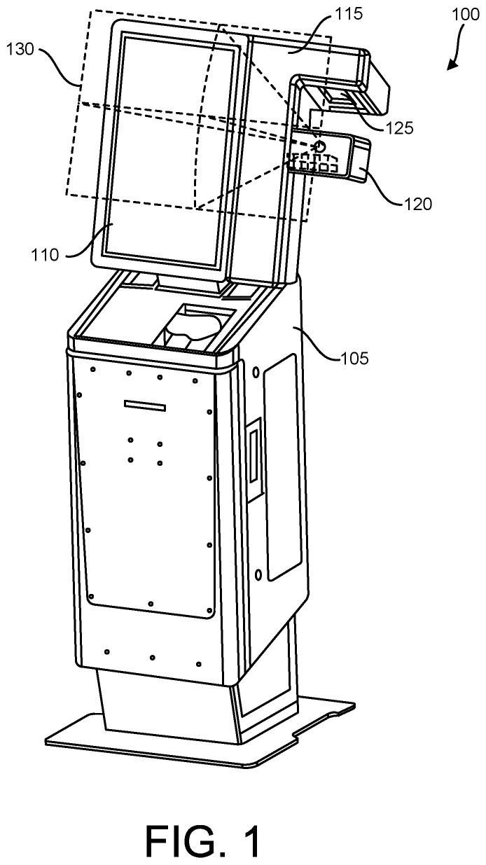

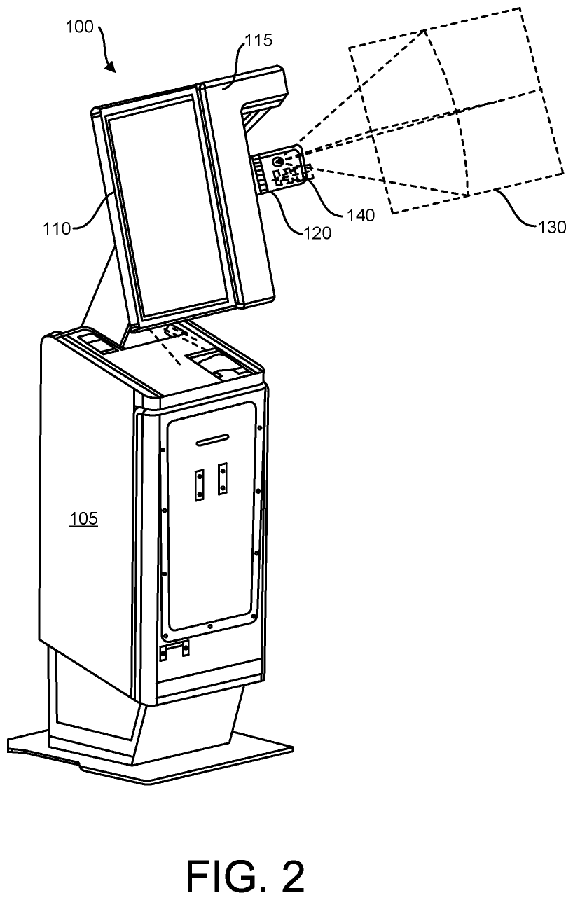

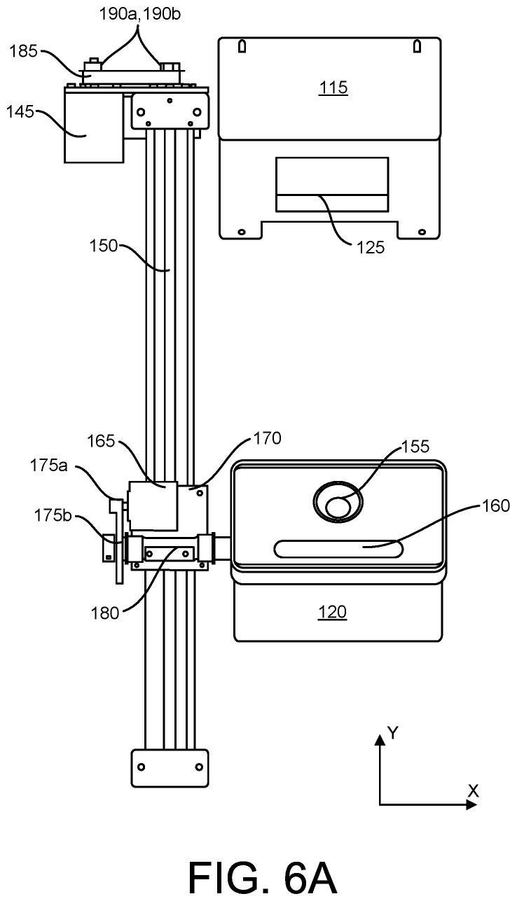

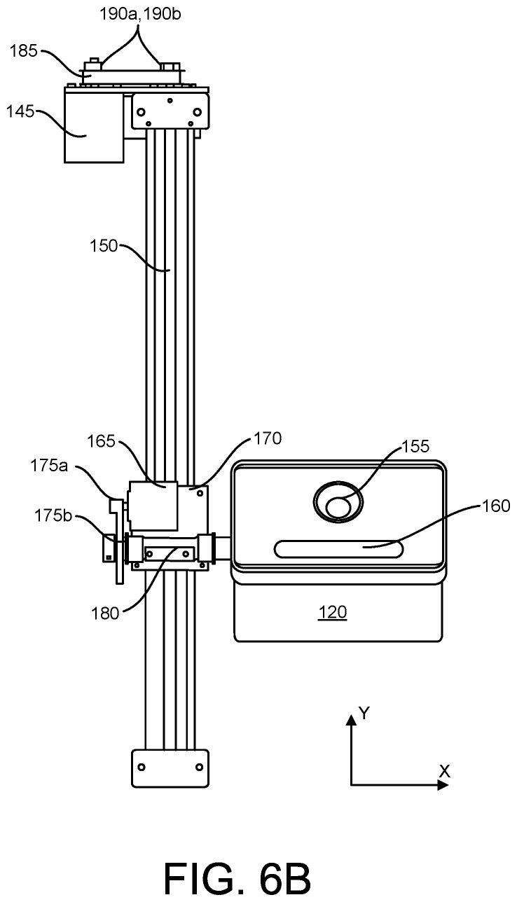

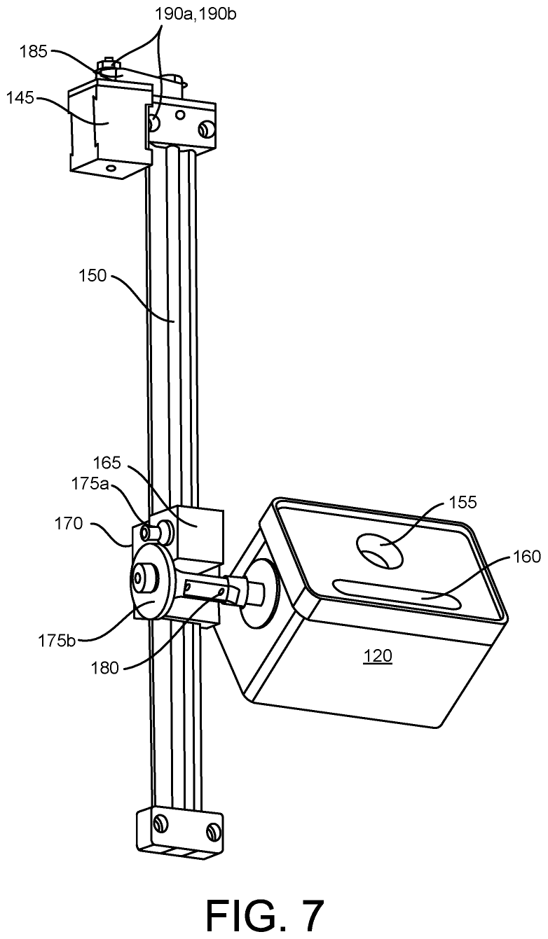

Referring to FIGS. 1 and 2, there is depicted a system 100 for scanning an individual's skin temperature in the form a temperature screening workstation. The system 100 or workstation may have a console 105 located at its bottom portion and a display 110 located above the console 105 at the top portion. Also included adjacent to the display 110 above the console 105 is a housing 115. The housing 115 contains and covers a blackbody 125; hence the blackbody 125 is coupled to or integrated within the housing, thereby minimizing the workstation's footprint and increasing the workstation's temperature measuring accuracy. The top portion of the workstation also includes a camera housing subassembly 120 coupled to the housing 115, wherein the camera housing subassembly 120 is moveable relative to the blackbody 125. As discussed in more detail below, particularly with respect to FIGS. 6A, 6B and 7, the camera housing subassembly 120 comprises a thermal imaging device 155, such as an IRT, and a proximity sensor 160.

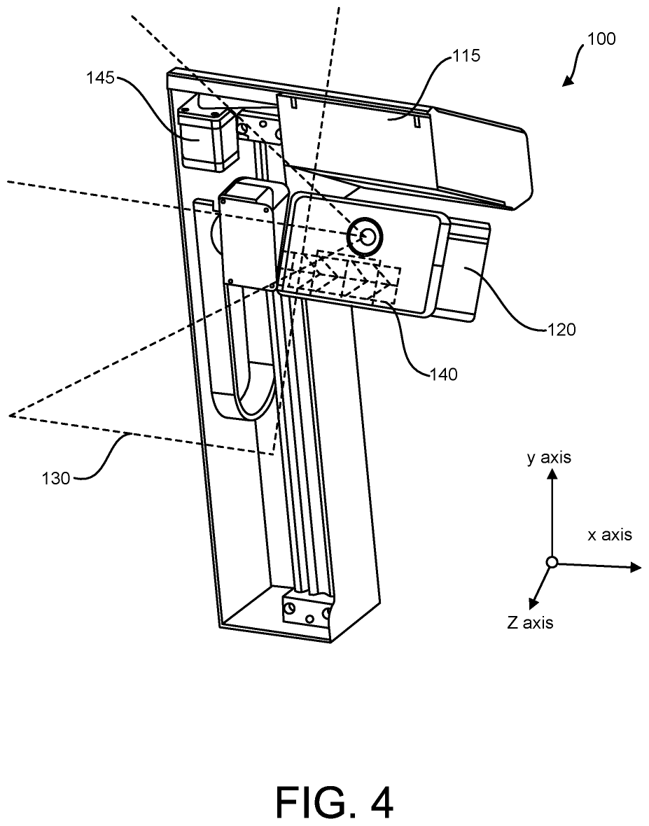

The thermal imaging device 155 is capable of moving relative to the blackbody 125. For example, the thermal imaging device 155 translates linearly both toward and away from the blackbody 125 along a vertical axis (i.e., y axis), which may be substantially and vertically aligned with the housing 115. The thermal imaging device 155 also rotates about a horizontal axis (i.e., x axis), which cuts through the center of the camera housing subassembly 120 from its left end to its right end. As shown in FIG. 4, the horizontal axis is offset substantially ninety (90) degrees from the vertical axis.

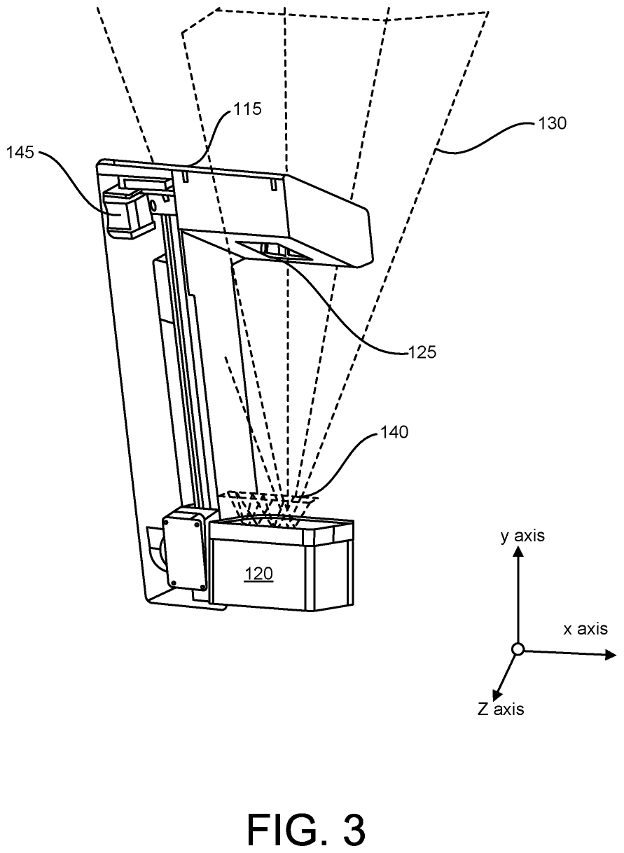

Referring to FIGS. 3 and 4, the camera housing subassembly 120 moves between a calibrated position and an individual image taking position. FIG. 3 depicts the camera housing subassembly 120 and the thermal imaging device 155 in a calibrated position, and FIG. 4 depicts the camera housing subassembly 120 and the thermal imaging device 155 in an individual image taking position. Referring to FIG. 3, when the camera housing subassembly 120 and the thermal imaging device 155 are in the calibrated position, the thermal imaging device 155 is substantially aligned with the blackbody 125 along a vertical axis and the thermal imaging device 155 faces the blackbody 125 such that the thermal imaging device 155 is focused on obtaining an image of the blackbody 125. The actual distance from the thermal imaging device 155 to the blackbody 125 can be closer than that is shown if FIG. 3. For example, depending upon the resolution of the IRT, it may be desirable for the camera to be positioned approximately between 5 and 10 inches (i.e., 5.0, 5.5, 6.0, 6.5, 7.0, 7.5, 8.0, 8.5, 9.0, 9.5 or 10.0) inches away from blackbody 125. Although the field of view 130 for the thermal imaging device 155 in the calibration position is adjustably focused at set up, the focus distance typically remains fixed after set up, although the scope of this disclosure envisions additional focusing capabilities during use after set up.

Referring to FIG. 4, when the camera housing subassembly 120 and the thermal imaging device 155 are in the individual image taking position, the thermal imaging device 155 may substantially aligned with the blackbody 125 along a vertical axis and the thermal imaging device 155 faces away from the blackbody 125 such that the thermal imaging device 155 is focused on obtaining an image of the individual person, particularly the person's head. Continuing to refer to FIG. 4, item 130 is the field of view for the thermal imaging device 155 when the thermal imaging device 155 is in the individual image taking position. For example, depending upon the resolution of the IRT, it may be desirable for the camera to be positioned approximately between 17 and 25 inches (i.e., 17.0, 17.5, 18.0, 18.5, 19.0, 19.5, 20.0, 20.5, 21.0, 21.5, 22.0, 22.5, 23.0, 23.5, 24.5 or 25.0) inches away from the subject's face to meet the FDA target zone requirement of 240 mm height×180 mm width for a subject's facial size, where 1 pixel is equal to or about 1 mm. Although the field of view 130 for the thermal imaging device 155 in the individual image taking position is adjustably focused at set up, the focus distance typically remains fixed after set up, although the scope of this disclosure envisions additional focusing capabilities during use after set up.

The thermal imaging device 155 has the same or similar number of pixels whether taking an image of the blackbody 125 and/or subsequently or previously taking an image of the person. As will be discussed in more detail below, the frame of the image of the blackbody 125 is smaller than the size a human face. By moving the imaging device 155 closer to the blackbody 125 in relative comparison to the subject, the blackbody 125 encompasses more of the imaging device's field of view that it would for a similarly sized blackbody located at the same distance as the subject from the imaging device 155. That is, by locating the blackbody 125 closer to the imaging device 155, the blackbody, which is smaller than the subject's face, can fill the same pixel area as that of the subjects face.

Continuing to refer to FIGS. 3 and 4, item 140 is the field of view for the proximity sensor, which is disposed in the camera housing subassembly 120.

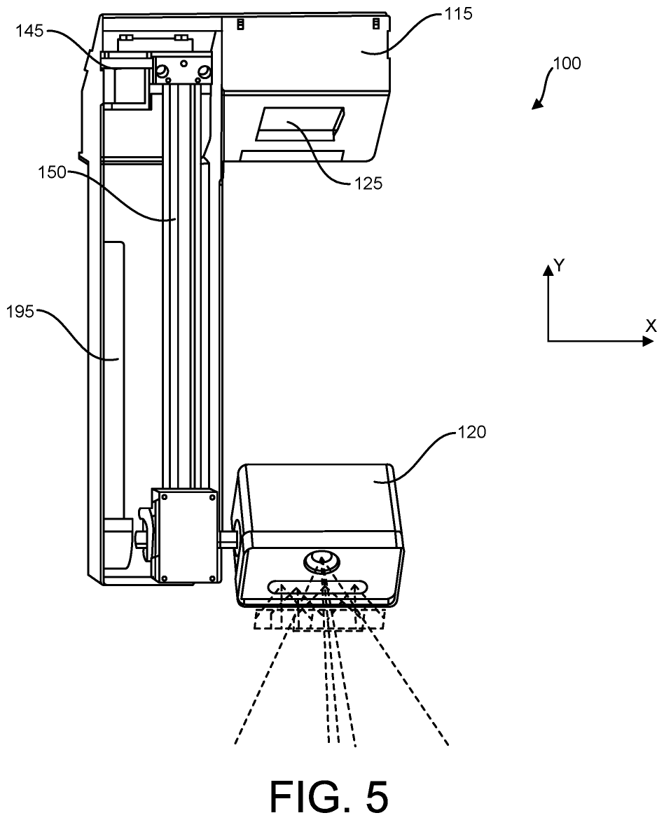

Referring to FIGS. 5, 6A, 6B and 7, there is depicted a motorized drive system configured to move the camera housing subassembly 120 and the thermal imaging device 155 between the calibrated position and the individual image taking position, as well as to and/or from and between any other position(s). The motorized drive system may include one motor and drive system to linearly translate the camera housing subassembly 120 and the thermal imaging device 155 toward and away the blackbody along a vertical axis (i.e., y axis) and another motor and drive system to rotate the camera housing subassembly 120 and the thermal imaging device 155 about a horizontal axis (i.e., x axis), wherein the horizontal axis is offset substantially ninety (90) degrees from the vertical axis.

The motor and drive system that linearly translates the camera housing subassembly 120 and the thermal imaging device 155 toward and away the blackbody along a vertical axis may include (i) a motor 145, (ii) a linear drive rod 150, such a threaded rod, coupled to the motor 145 via a plurality of mechanical coupling components, such as a belt 185 and pulleys 190a, 190b, (iii) a carriage 170 connecting the linear drive rod 150 to the camera housing subassembly 120 and (iv) a plurality of travel sensors to ensure that the motor 145 rotation and/or carriage 170 travel is limited. The carriage 170 may include a linear slide, which is coupled to and cooperates with the linear drive rod 150. As the motor 145 rotates, the linear drive rod 150 rotates, thereby driving the carriage 170 (via linear movement of the linear slide) and the camera housing subassembly 120 along the vertical axis.

The motor and drive system that rotates the camera housing subassembly 120 and the thermal imaging device 155 about the horizontal axis may be coupled to and/or included within the carriage 170. For example, the motor and drive system that rotates the camera housing subassembly 120 and the thermal imaging device 155 about the horizontal axis may include (i) a motor 165, (ii) a drive shaft 180 coupled to the motor 165 via a plurality of mechanical coupling components, such as a set of gears 175a, 175b, and (iii) a plurality of travel sensors to ensure that the motor 165 rotation and/or camera housing subassembly 120 and thermal imaging device 155 rotation is limited. As the motor 165 rotates, the gears 175a, 175b mesh and rotates, thereby driving the rotation of the drive shaft 180, the camera housing subassembly 120 and the thermal imaging device 155 about the horizontal axis because the drive shaft 180 is directly connected to the camera housing subassembly 120.

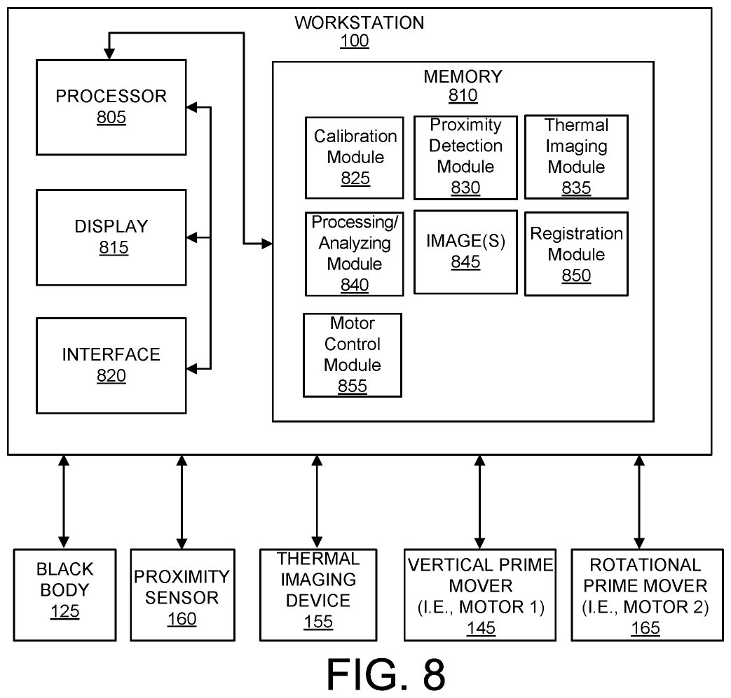

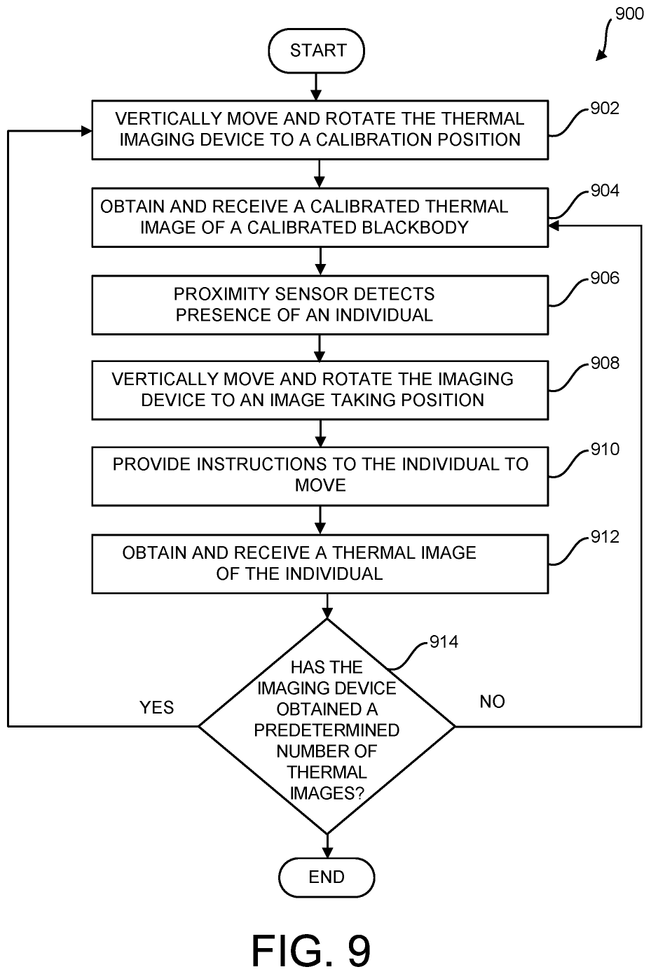

Referring to FIG. 8, there is depicted an illustration of an exemplary block diagram of a workstation 100 or console having a control system from which an individual person may use to implement all or certain or a combination of the methods illustrated in FIG. 9. The workstation 100 preferably includes a computer system comprising one or more processors 805 and memory 810 for storing programs and applications to perform the methods disclosed herein. Memory 810 may store a calibration module 825, a proximity detection module 830, a thermal scanning module 835, a processing/analyzing module 840, images 845, a registration module 850, and a motor control module 855. The workstation 800 may also include a display 815 for viewing the images 845 of the individual people. Display 815 may also permit a user to interact with the workstation 800 and its components and functions (e.g., touchscreen, graphical user interface, etc.), or any other element within the system. This is further facilitated by an interface 820 which may include a keyboard, mouse, a joystick, a haptic device, or any other peripheral or control to permit user feedback from and interaction with the workstation 800.

The workstation 800 may also include or be coupled to the blackbody 125, the proximity sensor 160, the thermal imaging device 155, a vertical prime mover (e.g., motor) 145 and a rotational prime mover (motor) 165. The calibration module 825 may be configured to have the camera housing subassembly 120 and the thermal imaging device 155 move to a calibrated position and have the thermal imaging device 155 obtain a thermal image of the blackbody 125 when the thermal imaging device 155 is in a calibrated position. The thermal imaging module 830 may be configured to receive signals from the calibration module 825 and/or the proximity detection module 830. In that regard, all modules may be logically coupled such that they work together and are coordinated.

For example, when the calibration module 825 confirms that the camera housing subassembly 120 and the thermal imaging device 155 are located in a calibrated position, the calibration module 825 may communicate with the thermal scanning module 835, which instructs the thermal imaging device 155 to obtain a thermal image of the blackbody 125. As another example, when the proximity detection module 830 and/or the proximity sensor 160 confirms the presence of an individual, the individual is accurately located within the thermal imaging device's 155 field of view, and/or the camera housing subassembly 120 and the thermal imaging device 155 are located in individual image taking position, the proximity detection module 830 may communicate with the thermal scanning module 835, which instructs the thermal imaging device 155 to obtain a thermal image of the individual.

The motor control module 855 communicates with a motorized drive system configured to move the camera housing subassembly 120 and the thermal imaging device 155 between the calibrated position and the individual image taking position, as well as to and from and between any other position(s). That is, the motor control module 855 communicates with and instructs the motor 145 to drive the drive system that linearly translates the camera housing subassembly 120 and the thermal imaging device 155 toward and away the blackbody along the vertical axis (i.e., y axis). The motor control module 855 also communicates with and instructs the motor 165 to drive and drive system to rotate the camera housing subassembly 120 and the thermal imaging device 155 about the horizontal axis (i.e., x axis). The motor control module 855 also communicates with and receives signals from the travel sensors to ensure that the rotation of the respective motors 145, 165 and/or camera housing subassembly 120 and thermal imaging device 155 rotation or linear travel is limited.

The thermal images 845 taken by the thermal imaging device 155, such as images of the blackbody 125 and the individual, are stored in the memory 810. The registration module 850 coordinates the stored images of the blackbody 125 and the individual. And the processing/analyzing module 840 analyzes the image(s) of the individual(s), and if desirable, calibrates such images using the associated images of the blackbody 125.

Referring to FIG. 9, there is depicted a flow diagram of an example of a method 900 of operating the workstation 100, in accordance with the present disclosure. That is, the workstation 100 operates the thermal imaging device 155, and other components of the workstation 100, according to this method 900 in order to obtain individuals' skin temperature to screen individuals with an elevated skin temperature. Additionally, and/or alternatively, a non-transitory computer-readable medium (e.g., memory within the computing system of the workstation 100) may include instructions that when executed by one or more processors (e.g., processors within the computing system) may cause the processors to perform the steps of the method 900.

Step 902 may comprise moving the thermal imaging device 155 to a calibrated position. For example, step 902 may include linearly translating the thermal imaging device 155 toward the blackbody 125 along the vertical axis and rotating the thermal imaging device 155 about the horizontal axis to a calibrated position. Step 904 may comprise having the thermal imaging device 155 obtain a thermal image of the blackbody 125 when the thermal imaging device 155 is in the calibrated position, such as when the thermal imaging device 155 is directed toward the blackbody 125. The size of the thermal image of the blackbody 125 will be predetermined because the thermal imaging device 155 will be focused to image the blackbody 12 at a predetermined distance from the thermal imaging device 155. For example, depending upon the resolution of the IRT, it may be desirable for the thermal imaging device 155 to be positioned at approximate distance between 7.5 inches and 8 inches from the blackbody 12. Based on this positioning, 1 pixel is equal to or about 1 mm, and the thermal imaging device 155 obtains a 3 inch by 3 inch sized image of the blackbody, wherein the image has a resolution of about 240×240 pixels. This image may be referred to as a blackbody image. That is, the frame size of the blackbody image is 3 inches by 3 inches.

The blackbody typically includes a USB communication interface that allows for the following functions: (i) setting the blackbody temperature range between 30° C. and 45° C. (86° F. to 113° F.)—setting must be above ambient temperature; (ii) getting the reference temperature setpoint; (iii) getting the current reference temperature; (iv) getting the ambient temperature; (v) getting the ambient humidity; (vi) getting the status of the device, (ready, busy, error): (vii) getting the serial number of the device; (viii) turn the blackbody device on-off. These communication features allow for remote setting and control of both the blackbody for engineering testing to gather IRT temperatures at various distances for different black body temperature settings, the effect ambient temperature and humidity have on the temperature readings of the IRT as well as determining when new reference temperatures need to be taken. The blackbody temperature reference should be set to a relatively high temperature threshold above ambient temperature, such as between 30° C. and 45° C. (86° F. to 113° F.), thereby providing a sufficient offset between the subject's skin temperature and the blackbody temperature reference. That is, the blackbody image should be obtained when the blackbody temperature reference should is set to a relatively high temperature threshold above ambient temperature.

Step 906 may comprise the proximity sensor 160 sensing or detecting the presence of an individual person approaching, proximate to and/or at the individual image taking position. Upon the proximity sensor 160 sensing or detecting the presence of an individual person, the method 900 performs the remaining steps. If, however, there has been a predetermined amount of time that has elapsed since the thermal imaging device 155 obtained a thermal image of the blackbody 125 in the calibrated position prior to the proximity sensor 160 sensing or detecting the presence of an individual person, steps 902 and/or 904 may need to be repeated before step 908 and any subsequent steps are performed.

Step 908 may comprise moving the thermal imaging device 155 to an individual image taking position. For example, the thermal imaging device 155 may move away from the blackbody 125 along the vertical axis and/or rotate about the horizontal axis to the individual image taking position. That is, the thermal imaging device 155 automatically adjusts its field of view to the height of the individual and thermal imaging device 155 focuses the individual's face. The display 110 provides an outline for the face and a second outline for the target area between the eyes of the subject. The system and method 900 then determines that the subject's face is free of obstructions, such as a mask or glasses and automatically senses the temperature in the region medial to the inner canthus of the eye(s). Depending upon the individual's physical position relative to the thermal imaging device's 155 field of view, the method 900 may include step 910, which may comprise instructing the individual to move relative to the thermal imaging device 155 and/or allowing the individual to move the thermal imaging device 155 to the individual image taking position using the display 815 and/or the interface 820.

Step 912 may comprise having the thermal imaging device 155 obtain a thermal image of the individual person, particularly the person's head, face, neck and/or shoulders, when the thermal imaging device 155 is in the individual image taking position, such as when the thermal imaging device 155 is directed toward the person's head, face, neck and/or shoulders. Upon obtaining a thermal image of the individual person, the person's skin temperature (e.g., at the respective portions of the person's head, face, neck and/or shoulders) is calculated and provided to the person as an output via the display 815 and/or the interface 820. The person's skin temperature is calculated based at least in part as a function and/or with reference to the thermal image of the blackbody 125 previously obtained in step 904 above.

As discussed above, the blackbody image, which is taken when the thermal imaging device 155 is in a calibrated position, has a frame size of about 3 inches by 3 inches with a resolution of about 240×240 pixels. The image of the subject, which is taken when the thermal imaging device 155 is in a image taking position, has a frame size of about 5.7 inches by 8.7 inches with a resolution of about 180×240 pixels. Moreover, rotating the imaging device 155 allows such device to cover the whole vertical range of 240×512 pixels. Because the distance from the thermal imaging device 155 to the blackbody 125 is different (i.e., less than) the distance from the thermal imaging device 155 to the subject and the imaging device 155 can be adjusted such that the imaging device 155 senses and images the region of the head located medially to the inner canthus of the eye.

Referring again to FIG. 9, the same calibration determination may be re-used when determining the temperature of additional individuals before repeating the calibration step 902. For example, method 900 may include step 914, which determines whether the imaging device 155 has obtained a predetermined number of images when the imaging device 155 is in the image taking position after obtaining each thermal image. If the imaging device 155 has obtained a predetermined number of such images, step 902 is repeated before obtaining additional thermal images. If the imaging device 155 has not obtained a predetermined number of such images, step 904 (obtaining additional thermal images) is repeated without first repeating step 902. The present disclosure, therefore, encompasses using a single blackbody image in calculating the temperature of a plurality of different individuals. Rather than step 914 being based on the predetermined number of thermal images obtained, step 914 may be based on the amount of a predetermined amount of time elapsing since a thermal image was obtained, a certain change in ambient air temperature compared to the ambient temperature of last blackbody image, a certain change in ambient air humidity in comparison to the ambient air humidity of last blackbody image, any combination of the foregoing, etc.

Although it is not shown in FIG. 9, the method may also include the step of comparing the person's highest skin temperature, of the scanned region, to a predetermined acceptable and/or unacceptable temperature range and outputting to the display 815 and/or the interface 820 whether the person's skin temperature is acceptable and/or unacceptable, thereby allowing the systems and methods discussed herein to screen people efficiently for an elevated body temperature. For example, the system and method provide the workstation 100 the ability to output the testing/screening results in multiple formats including, but not limited to, a display screen message, a printed message and a digital data packet that can then be utilized by other software systems such as a building security system.

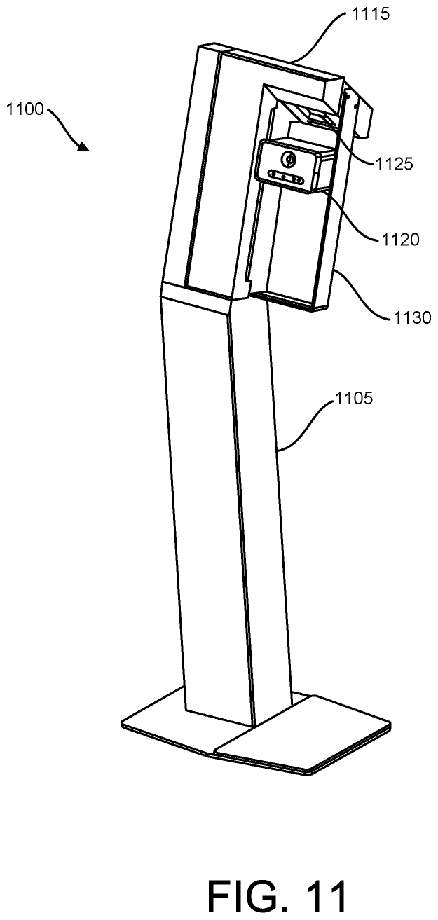

Referring to FIG. 11, there is depicted a system 1100 for scanning an individual's skin temperature in the form a temperature screening workstation. The system 1100 is similar to the system 100 described above. That is, generally, the system 1100 may have a console 1105 located at its bottom portion, and included above the console 1105 is a housing 1115. The housing 1115 contains and covers a blackbody 1125; hence the blackbody 1125 is coupled to or integrated within the housing 1115, thereby minimizing the workstation's footprint and increasing the workstation's temperature measuring accuracy. The top portion of the workstation also includes a camera housing subassembly 1120 for scanning an individual's skin temperature. The camera housing subassembly 1120 is moveable relative to the blackbody 1125. In contrast to the system 100, the system 1100 lacks a display and includes an additional support bracket 1130 for the camera housing subassembly 1120.

In some embodiments and as shown in FIG. 11, the console 1105 of the system 1100 may be constructed as a totem-like structure. In other embodiments, the console 1105 may be constructed in other forms (for example, a shorter structure configured to be positioned on a counter or table), or the console 1105 may be omitted. In some embodiments, the system 1100 may be wall mounted. In some embodiments, the remainder of the system 1100 may be a module that is selectively mountable to a console 1105 or a wall.

Various modifications and additions can be made to the exemplary embodiments discussed without departing from the scope of the present disclosure. That is, while the embodiments described above refer to particular features, the scope of this disclosure also includes embodiments having different combinations of features and embodiments that do not include all the described features. The blackbody may be mounted onto a second motorized system that moves the blackbody into view of the imaging device. Additionally, alternative mounting locations for the blackbody may exist such that the blackbody may be mounted to the enclosure but positioned in front of the imaging device so that instead of the imaging device rotating to look at the blackbody, the imaging device would move vertically while looking forward. As another alternative, a blackbody may be provided separately from a housing. As a more specific example and referring to FIGS. 1 and 2, the blackbody 125 may not be coupled to or integrated within the housing 115. Furthermore, although the present disclosure discusses linearly moving the imaging device with one motor and rotating the imaging device with another motor, this disclosure envisions using a single motor and cam assembly to translate and rotate the imaging device through a cam path motion rather than using a second motor. Accordingly, the scope of the present disclosure is intended to embrace all such alternatives, modifications, and variations as fall within the scope of the claims, together with all equivalents thereof.

What is claimed is:

1. A system for scanning an individual's skin temperature, the system comprising:
a. a housing;
b. a blackbody;
c. a camera housing subassembly coupled to the housing, wherein the camera housing subassembly is moveable relative to the blackbody, wherein the camera housing subassembly comprises a proximity sensor and a thermal imaging device;
d. a motorized drive system coupled to the camera housing subassembly, wherein the motorized drive system is configured to (i) move the thermal imaging device toward and away the blackbody along a vertical axis and (ii) rotate the thermal imaging device about a horizontal axis, wherein the horizontal axis is offset substantially 90 degrees from the vertical axis; and
e. non-transitory computer readable medium having a computer program stored thereon for controlling movement of the thermal imaging device and timing at which the thermal imaging device obtains a thermal image of the individual's skin temperature, the computer program comprising instructions for causing one or more processors to:
i. move the thermal imaging device toward the blackbody along the vertical axis and rotate the thermal imaging device about the horizontal axis to a calibrated position;
ii. obtain a first thermal image when the thermal imaging device is in the calibrated position and directed toward the blackbody;
iii. move the thermal imaging device away from the blackbody along the vertical axis and rotate the thermal imaging device about the horizontal axis to an individual image taking position;
iv. obtain a second thermal image when the thermal imaging device is in the individual image taking position and directed toward the individual; and
v. calculating the individual's skin temperature as a function of the second thermal image and the first thermal image.

2. The system of claim 1, wherein the computer program further comprises instructions to output the individual's skin temperature.

3. The system of claim 1, wherein the motorized drive system is configured to linearly translate the thermal imaging device toward and away the blackbody along the vertical axis.

4. The system of claim 3, wherein the computer program further comprises instructions for causing one or more processors to linearly translate the thermal imaging device toward the blackbody along the vertical axis to the calibrated position.

5. The system of claim 3, wherein the computer program further comprises instructions for causing one or more processors to linearly translate the thermal imaging device away from the blackbody along the vertical axis to the individual image taking position.

6. The system of claim 1, wherein the computer program further comprises instructions to calculate the individual's skin temperature as a function of the second thermal image being calibrated by the first thermal image.

7. The system of claim 1, wherein the motorized drive system comprises:
a. a first motor;
b. a threaded rod coupled to the first motor; and
c. a linear slide coupled to the threaded rod and camera housing subassembly, whereupon rotation of the first motor, the threaded rod rotates and the linearly translate the camera housing subassembly toward or away from the blackbody along the vertical axis.

8. The system of claim 1, wherein the motorized drive system comprises:
a. a second motor; and
b. a drive train coupled to the second motor and the camera housing subassembly, whereupon rotation of the second motor, the camera housing subassembly rotates about the horizontal axis.

9. The system of claim 1, wherein the blackbody is coupled to the housing.

10. The system of claim 1, wherein the blackbody is separate from the housing.

11. A method for scanning an individual's skin temperature, the method comprising:
a. providing a skin temperature sensing system, the system comprises:
i. a housing;
ii. a blackbody coupled to the housing;
iii. a camera housing subassembly coupled to the housing, wherein the camera housing subassembly is moveable relative to the blackbody, wherein the camera housing subassembly comprises a proximity sensor and a thermal imaging device; and
iv. a motorized drive system coupled to the camera housing subassembly, wherein the motorized drive system is configured to (i) move the thermal imaging device toward and away the blackbody along a vertical axis and (ii) rotate the thermal imaging device about a horizontal axis, wherein the horizontal axis is offset substantially 90 degrees from the vertical axis; and
b. moving the thermal imaging device toward the blackbody along the vertical axis and rotating the thermal imaging device about the horizontal axis to a calibrated position;
c. obtaining a first thermal image when the thermal imaging device is in the calibrated position and directed toward the blackbody;
d. moving the thermal imaging device away from the blackbody along the vertical axis and rotating the thermal imaging device about the horizontal axis to an individual image taking position;
e. obtaining a second thermal image when the thermal imaging device is in the individual image taking position and directed toward the individual; and
f. calculating the individual's skin temperature as a function of the second thermal image and the first thermal image.

12. The method of claim 11, further comprising displaying the individual's skin temperature.

13. The method of claim 11, wherein the motorized drive system is configured to linearly translate the thermal imaging device toward and away the blackbody along the vertical axis.

14. The method of claim 12, wherein moving the thermal imaging device comprises linearly translating the thermal imaging device toward the blackbody along the vertical axis to the calibrated position.

15. The method of claim 12, wherein moving the thermal imaging device comprises linearly translating the thermal imaging device toward the blackbody along the vertical axis to the individual image taking position.

16. The method of claim 11, further comprising calculating the individual's skin temperature as a function of the second thermal image being calibrated by the first thermal image.
